(19) United States
(12) Patent Application Publication
(10) Pub. No.: US 2025/0366835 A1
(43) Pub. Date: Dec. 4, 2025

(54) CLEANING OF A URINE ANALYSIS DEVICE

(71) Applicant: Withings, ISSY LES MOULINEAUX (FR)

(72) Inventor: Arthur PONS, ISSY LES MOULINEAUX (FR)

(21) Appl. No.: 19/219,501

(22) Filed: May 27, 2025

(30) Foreign Application Priority Data
May 30, 2024 (FR) FR2405625

(51) Int. Cl. A61B 10/00; E03D 11/00

(52) U.S. Cl. CPC A61B 10/007; E03D 11/00

(57) ABSTRACT

A urine analysis assembly includes a urine analysis device configured to be placed entirely within a toilet bowl and including a housing, the housing including a front face for receiving a stream of urine directly from a user urinating into the toilet bowl, a fastening arm configured to position the urine analysis device entirely within the toilet bowl, the fastening arm including a water supply ramp configured to direct a flow of water from a flush of the toilet bowl towards the front face of the housing.

### CROSS-REFERENCE TO RELATED APPLICATIONS

This application claims priority to French Patent Application No. FR2405625, filed May 30, 2024, the entire content of which is incorporated herein by reference in its entirety.

### FIELD

The present invention relates to a urine analysis assembly comprising a urine analysis device. The urine analysis device comprises a housing configured to be placed entirely within a toilet bowl.

### BACKGROUND

Many biological parameters can be identified in an individual's urine. For example, health problems such as urinary tract infection, diabetes or kidney failure can be detected from a urine sample. The urine sample can also reflect the quality of a diet, identify a period of fertility or pregnancy, and detect drug or tobacco use. It therefore makes sense to periodically monitor various biological parameters.

It is known to offer toilet devices with a urine analysis function. These devices are capable of taking urine samples from the toilet and analyzing them to determine the level of a biological parameter.

Such a urine analysis device is described in particular in document WO2021/175909. This document discloses a device for analyzing urine. The device fits into a toilet and collects a urine sample prior to optical analysis. The device comprises a station and a cartridge, also known as a rotary holder, which can be removed and replaced from the station. The cartridge contains urine strips, i.e. strips coated or impregnated with a reagent that reacts with urine.

Such a urine analysis device placed in a toilet bowl faces the problem of cleaning the housing after a user has urinated on it. The toilet bowl has a flush mechanism that causes water to flow along the bowl. This flow of water tends to pass over and/or behind the housing, and therefore does not clean the front of the housing, which can present comfort and hygiene problems (yellowing color of the housing, odor, etc.) for the user.

### SUMMARY

The present description therefore proposes a urine analysis set that enables the urine analysis device to be cleaned after a user has urinated.

To this end, the present description relates to a urine analysis assembly comprising:
a urine analysis device configured to be placed entirely within a toilet bowl and comprising a housing, the housing comprising a front face for receiving a stream of urine directly from a user urinating into the toilet bowl,
a fastening arm configured to position the urine analysis device entirely within the toilet bowl, the fastening arm comprising a water supply ramp configured to direct a flow of water from a toilet bowl flush towards the front face of the housing.

In fact, this type of urine analysis assembly makes it possible to provide a urine analysis device located in a toilet, and therefore subject to numerous user micturitions on the front face, which can be cleaned at least in part by means of the fastening arm. In fact, the water supply ramp on the fastening arm enables a flow of water from the flush to be directed towards the front face of the housing, thereby cleaning this front face in part. The directed flush cleaning via the ramp maintains hygiene and aesthetic appearance over extended use periods, reducing the need for manual cleaning. This is especially relevant in multi-user or clinical environments where device appearance and cleanliness directly impact adoption. The fastening arm as described herein provides a spatially offset and mechanically stable configuration that avoids interference with irregular toilet bowl geometries (e.g., shoulders, contours, or projections), thereby improving compatibility, durability of attachment, and accuracy of urine collection due to optimal sensor alignment. The receiving space extends from the rear face to beyond the upper edge of the housing, thereby partially enveloping the housing's upper contour. This configuration not only stabilizes the housing within the toilet bowl but also mechanically restricts rotational and translational displacements under lateral forces or flushing turbulence.

In an embodiment, the fastening arm is flexible. As used herein, the term “flexible” refers to the ability of the fastening arm to elastically deform in at least one direction (for example in the median plane XZ) in response to external forces such as manual installation pressure or the gravitational weight of the urine analysis device. Such flexibility allows the arm to conform to variable toilet geometries without permanent deformation, enabling a resilient fit that ensures proper positioning and reliable contact of the housing with the inner wall of the toilet. In some embodiments, flexibility may result from the use of polymeric materials (e.g., nylon, polyethylene) or thin metal structures with an elastic modulus suitable for temporary deflection.

In an embodiment, the fastening arm is removable.

In an embodiment, the fastening arm has a flattened shape in a section transverse to a median plane of the device. As used herein, the term “flattened” refers to a cross-sectional geometry in which one dimension (e.g., height along the Z axis) is substantially greater than the other (e.g., thickness along the Y axis), such that the section has an oblong, oval, or generally plate-like appearance. This geometry imparts directional flexibility to the fastening arm-allowing it to bend more easily within the median plane XZ-while maintaining lateral stiffness in the transverse direction Y. This controlled anisotropic flexibility improves both adaptability to curved toilet surfaces and mechanical stability.

In an embodiment, the fastening arm has a width of between 0.5 cm and 20 cm.

In an embodiment, the ramp extends over a length of between 1 cm and 20 cm.

In an embodiment, the ramp extends as an extension of the front face of the urine analysis device housing or overhangs the front face.

In an embodiment, the ramp is flush with the front face.

In an embodiment, the ramp extends from the housing towards the bowl, and forms an angle of between 0° and 90° with a plane tangent to the front face, particularly between 30° and 60°.

In an embodiment, the ramp is longitudinally convex and forms a springboard for the water.

In an embodiment, the ramp is transversely convex and forms a channel for the water.

In an embodiment, the ramp has at least one fin configured to extend towards the toilet bowl.

In an embodiment, the ramp has lateral fins.

In an embodiment, the ramp comprises a diffuser for flush water, the diffuser being configured to disperse water from the ramp water stream onto the front face of the housing.

In an embodiment, the diffuser is positioned on a transfer section at the junction with the housing.

In an embodiment, the diffuser comprises grooves.

In embodiments where the ramp includes a diffuser or lateral fins, water dispersion is enhanced, increasing the effective surface area coverage of the flush and improving cleaning uniformity across the front face. Lateral fins are particularly beneficial in toilets with asymmetric flush patterns, enabling redirection of oblique water streams.

In an embodiment, the fastening arm comprises a proximal end portion configured to cooperate with the housing.

In an embodiment, the fastening arm further comprises a distal end portion configured to cooperate with the toilet bowl.

In an embodiment, the fastening arm further comprises an intermediate portion arranged between the proximal end portion and the distal end portion.

In an embodiment, the distal end portion comprises a hook/magnet/adhesive part.

In an embodiment, the ramp comprises a transfer section close to the housing and configured to promote the passage of water from the water flow of the ramp to the front face of the housing.

In an embodiment, the transfer section is flush with the front face of the housing.

In an embodiment, the diffuser is positioned at the decanting section.

In an embodiment, the ramp comprises a collection section configured to collect at least some of the flush water.

In an embodiment, the collection section is configured to be close to, beneficially in contact with, the inner wall of the toilet bowl.

In an embodiment, the ramp is arranged between the proximal end portion and the distal end portion.

In an embodiment, the ramp is part of the intermediate portion.

In an embodiment, the ramp is gutter-shaped.

In an embodiment, the ramp has a width greater than 1 cm.

In an embodiment, the fastening arm further comprises a distal end portion configured to cooperate with the toilet bowl and an intermediate portion arranged between the proximal end portion and the distal end portion, the intermediate portion comprising a connecting portion and the ramp.

In an embodiment, the connecting portion comprises a passage area for the passage of water from the water flow.

In an embodiment, the passage area is connected to the collection section.

In an embodiment, the collection section comprises a fin.

In an embodiment, the ramp is arranged between the proximal end portion and the distal end portion.

In an embodiment, the passage area is defined by an opening in the connecting portion.

In an embodiment, the passage zone is defined by a cross-sectional constriction at the connecting portion.

In an embodiment, the ramp is arranged between the proximal end portion and the connecting portion.

In an embodiment, the connecting portion comprises a passage area connected to the collection section of the ramp.

In an embodiment, the ramp and connecting portion form a U or V shape.

In an embodiment, the collection section is offset towards the bowl.

In an embodiment, the connecting portion is arranged between the transfer section and the distal end portion.

In an embodiment, the collection section of the ramp extends from a junction between the connecting portion and the transfer section, the collection section comprising a free end on the opposite side to the junction.

In an embodiment, the collection section comprises a fin.

In an embodiment, the connecting portion comprises a passage zone, from which the collection section of the ramp extends in the opposite direction to the transfer section.

In an embodiment, the collection section of the ramp protrudes from the passage area.

In an embodiment, the collection section comprises one or more fins extending laterally from the junction.

In an embodiment, the collection section comprises two fins extending on either side of the junction.

In an embodiment, the collection section comprises a funnel that extends around the junction.

In an embodiment, the urine analysis device comprises a collection port, the collection port being configured to collect liquid from outside the housing to inside the housing.

In an embodiment, the collection port is located on a rear face, opposite the front face.

In an embodiment, the rear face comprises at least one spacer projecting from the rear face and configured to contact the toilet bowl.

An aspect of the invention also relates to a fastening arm configured to position a urine analysis device entirely within a toilet bowl, the urine analysis device comprising a housing comprising a front face for receiving a flow of urine directly from a user urinating into the toilet bowl, the fastening arm comprising a water supply ramp configured to direct a flow of water from a toilet bowl flush towards the front face of the housing.

Another aspect of the invention also relates to a method of operating a urine analysis assembly in a toilet bowl comprising: (a) receiving a urine stream on a front face of a housing; (b) directing flush water over the front face using a ramp attached to a fastening arm; and (c) collecting a portion of the urine sample via a rear-facing port into an internal analyzer.

### BRIED DESCRIPTION OF THE DRAWINGS

Further features, details and benefits will become apparent from the detailed description below, and from an analysis of the appended drawings, in which:

FIG. 1 shows a simplified schematic representation of a urine analysis device installed in a toilet bowl,

FIG. 2 shows an exploded view of the urine analysis device, in which the station and cartridge are visible,

FIG. 3 shows a detailed view of a cartridge in an embodiment,

FIG. 4 shows a cross-sectional view of a cartridge and station according to one embodiment, at the location of an optical analyzer of the station,

FIG. 5 shows a side view of the urine analysis device installed in a toilet bowl with an fastening arm in a resting configuration,

FIG. 6 shows a side view of an analysis assembly as per FIG. 5 but with the arm in a deployed configuration,

FIG. 7 shows a perspective view (a) and a side view (b) of a fastening arm of the assembly of FIGS. 5 and 6,

FIG. 8 shows a partial side view (a) of a urine analysis device and a front view (b) of the fastening arm as illustrated in FIGS. 5 to 7,

FIG. 9 shows a perspective view of a variant of the fastening arm of the assembly shown in FIG. 5,

FIG. 10 shows a partial side view (a) of a urine analysis device and a front view (b) of a variant of the fastening arm of the FIG. 5 assembly,

FIG. 11 shows a partial side view (a) of a urine analysis device and a front view (b) of a variant of the fastening arm of the FIG. 5 assembly,

FIG. 12 shows a partial side view (a) of a urine analysis device and a front view (b) of a variant of the fastening arm of the FIG. 5 assembly,

FIG. 13 shows a partial side view (a) of a urine analysis device and a front view (b) according to another variant of the assembly fastening arm shown in FIG. 5,

FIG. 14 shows a partial side view (a) of a urine analysis device and a front view (b) according to another variant of the alternative design of the fastening arm of the FIG. 5 assembly,

FIG. 15 shows a partial side view (a) of a urine analysis device and a front view (b) according to an alternative embodiment of the fastening arm of the FIG. 5 assembly

FIG. 16 shows a partial side view (a) of a urine analysis device and a front view (b) of a variant of the other embodiment of the fastening arm of the assembly of FIG. 15.

FIG. 17 shows a partial side view (a) of a urine analysis device and a front view (b) of a further embodiment of the fastening arm of the FIG. 5 assembly.

### DETAILED DESCRIPTION

The present description presents various examples of a urine analysis device comprising a station and a cartridge as disclosed in documents WO2021/175909 and WO2021/175944, hereinafter referred to as WO′909 and WO′944. Variations of the stations are disclosed in documents WO2023036805, WO2023036806, WO2023036808, WO2023036809, hereinafter referred to as WO′80X.

The following paragraphs explain the general principle of a urine analysis device, but all the details of documents WO′909 and WO′933 (as well as all the above-mentioned PCT documents) are applicable.

### Orientation

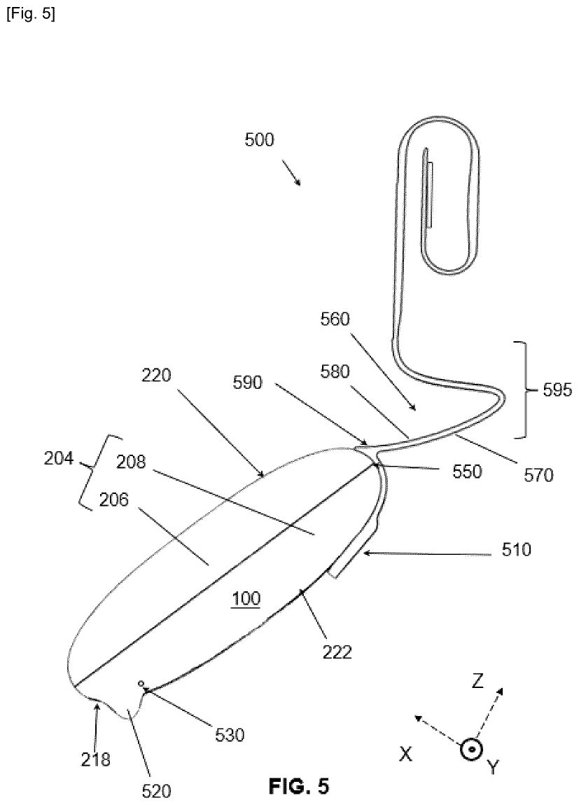

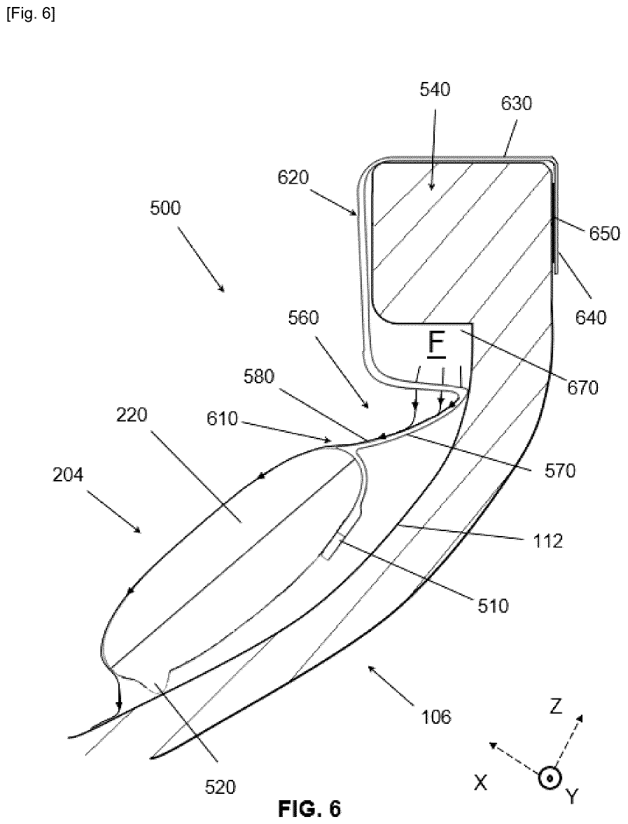

In the following, the notions of “top” and “bottom”, “upper” and “lower”, etc. are defined in relation to a Z direction, as defined in FIGS. 5 and 6. The top along the Z direction is defined in a normal use position of the urine analysis device fixed in the toilet bowl.

### Overall Shape of Housing

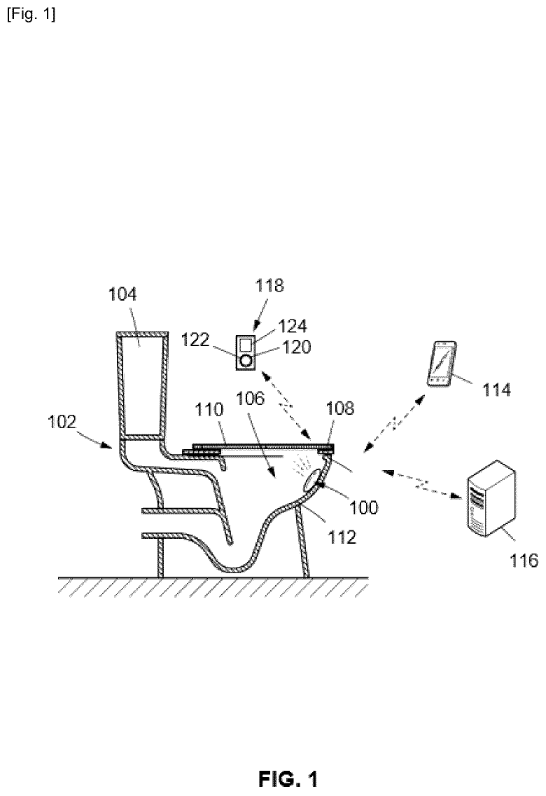

FIG. 1 schematically illustrates an analysis device 100 (referred to as “device 100”) for urine analysis installed in a toilet 102. Toilets 102 typically comprise a water tank 104, a bowl 106, a seat 108 and a seat cover 110. The analysis device 100 is configured to be placed entirely within the toilet bowl. By “in the bowl” is meant “placed in the interior volume defined by the bowl”. The analysis device 100 is removably arranged in the toilet 102. For example, the analysis device 100 can be easily removed from the toilet to replace a cartridge, then replaced in the 102 toilet. The analysis device 100 is placed on an inner wall 112 of the toilet bowl 106. The analysis device 100 is positioned so that it is generally under a user's urine stream, so that when a user urinates (generally in a seated position), the urine comes into contact with the analysis device 100. The analysis device 100 can communicate remotely with a remote entity, such as the smartphone 114 or a server 116.

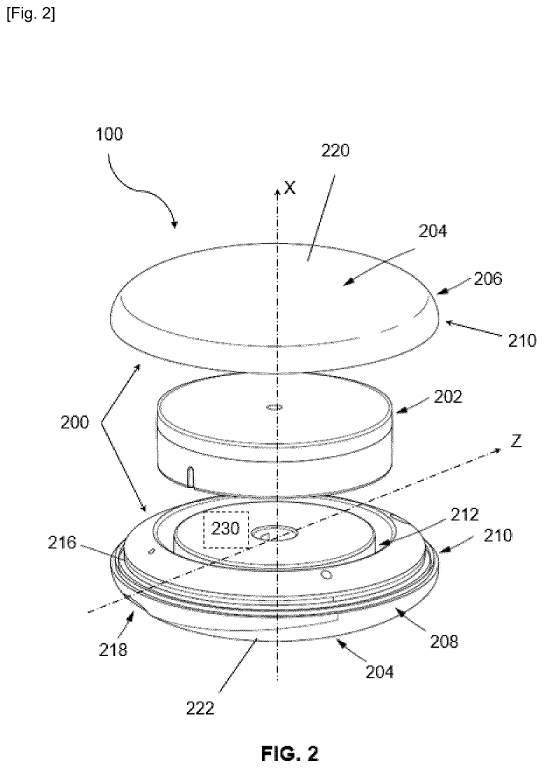

As illustrated in greater detail in FIG. 2, the analysis 100 device may comprise a station 200 and a cartridge 202, removably mounted on the station 200. The station 200 may comprise a housing 204 which may comprise two shells, in particular a front shell 206 and a rear shell 208. The front shell 206 and the rear shell 208 may cooperate with each other via a fastening mechanism 216, in a plane normal to the X axis. The front shell 206 and rear shell 208 can be reversibly assembled, for example by screwing or clipping. Alternatively, the front shell 206 and rear shell 208 can be permanently joined, for example by gluing, clipping, magnetizing or ultrasonic welding. Of course, other fastening methods can also be used to join the two shells together.

In particular, as shown in FIG. 2, the front shell 206 and the rear shell 208 are screwed together. Then, an internal part of the front shell 206 comprises a thread. The thread on the front shell 206 is designed to cooperate with a complementary thread on the rear shell 208. This allows the housing 204 to be easily disassembled to gain access to the test assembly inside the housing.

A seal may be present between the front shell 206 and the rear shell 208. In this way, the housing 204 is watertight. Only collection and drainage ports connect the outside and inside of housing 204, as described in more detail below.

As can be seen from the figures, the housing 204 may have the overall external shape of a circular roller. In other words, the housing 204 has a spheroidal shape. The X axis is the center line of the housing. Beneficially, the front shell 206 can be substantially rotationally symmetrical, giving the device an aerodynamic appearance once installed. Housing 204 serves as a urine collector.

The housing 204 comprises a front face 220 for receiving a stream of urine directly from a user urinating on the toilet, and a rear face 222 opposite the front face 220. As illustrated in FIG. 2, the front face 220 can be arranged on the front shell 206 and the rear face 222 can be arranged on the rear shell 208. The front face 220 faces the inside of the bowl 106. The front face 220 is therefore intended to receive urine when the user urinates while sitting on the toilet 102. As shown in FIGS. 5 and 6, the rear face 222 faces the inner wall 112 of the bowl 106. For the rest of the description, an object facing the bowl wall is taken to mean an object facing the bowl wall closest to the object in question, and not the opposite bowl wall on the other side of the inner bowl volume.

The front face 220 and rear face 222 each have a curved edge 210. The respective curved edges 210 of the front and rear faces meet at an equatorial junction zone. Thus, the outer surface of housing 204, consisting of front face 220 and rear face 222, is defined by curved lines and forms a generally convex object.

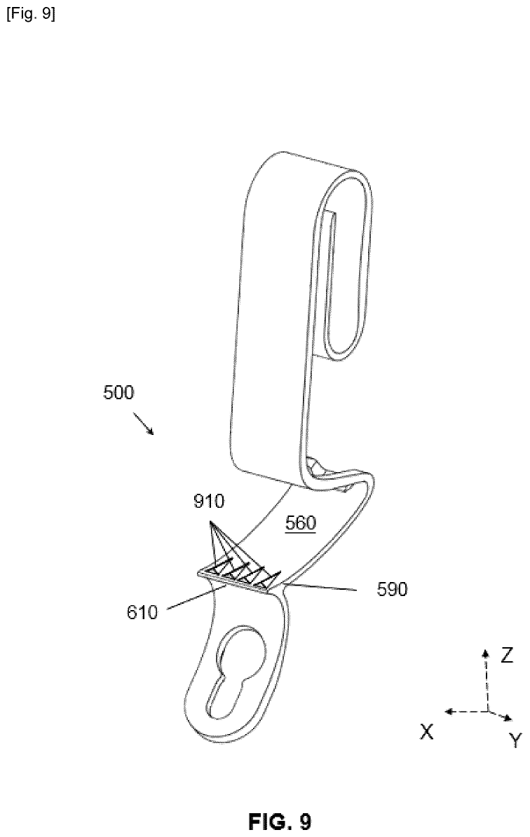

With reference to FIGS. 5 and 6, the housing 204 has an apex 550. The apex 550 is the highest point of the housing 204 along the Z axis in a normal use position of the housing 204 when placed in the toilet bowl 106. The apex 550 may be located at the junction between the front face 220 and the rear face 222. In an embodiment in which the housing 204 is parallelepipedal in shape, as shown in FIG. 9, the apex 550 is located on the upper face, at the junction between the front face 220 and the rear face 222.

The outer surface of the front face 220 may be smooth. In other words, the front face 220 is free of ridges or grooves. In this way, the flow of urine coming into contact with the front face 220 catches and spreads over the front face 220. The front face 220 can be substantially rotationally symmetrical about the X axis.

The outer surface of housing 204 can also be white or light-colored. The color of the outer surface can be similar to that of the toilet, which enhances the discreetness of the device.

Housing 204 can have a diameter, measured in the direction orthogonal to axis X, of between 50 mm and 150 mm. The housing 204 can have a thickness, measured in the direction of the X axis, of between 15 mm and 50 mm. In this way, housing 204 is sufficiently compact to be fully housed in the toilet bowl. The 100 urine analysis device is unobtrusive. In addition, housing 204 is large enough to systematically come into contact with the urine received in the toilet bowl. The user can then urinate in the toilet without worrying about the urine analysis device, or alternatively aim summarily.

According to another aspect, in an embodiment, the housing 204 has a general form factor such that the ratio between thickness and diameter is between 0.2 and 0.5, and even more preferably between 0.3 and 0.4. Such proportions are reminiscent of a natural pebble and give the device a soothing appearance. The spheroidal pebble shape minimizes splash-back and offers low resistance to water flow, encouraging complete and uniform flushing.

Preferably, housing 204 is made of a hydrophilic material. For example, the material of housing 204 may be: ceramic, polyamide (PA), silicone or a hydrophilic polymer. The outer surface of housing 204 can also be treated with a hydrophilic surface treatment, such as AcuWet® from Aculon, a hydrophilic polymer, or Pebax® from Arkema.

### Test Set

A test assembly 230 is located inside housing 204 and configured to perform an analysis on urine collected through the collection port. Station 200 comprises an annular compartment 212, located inside housing 204, arranged around an axis of rotation X. The annular compartment 212 is configured to at least partially receive the cartridge 202 mounted rotatably around the axis of rotation X (once in position in the annular compartment 212). The cartridge 202 comprises a plurality of test supports, each of which comprises at least one urine reagent, for example a dry reagent, the plurality of test supports being arranged along a circle or arc of a circle around the axis of rotation X. In an embodiment, the test supports are test strips. The test supports can be enclosed, for example individually, in a chamber.

The annular compartment 212 typically extends over 360° and forms a groove configured to at least partially receive the cartridge 202.

Station 200 comprises a collection port 218, located for example on the rear shell 208. As will be explained in more detail below, the collection port 218 is configured to collect urine flowing over the surface of the housing 204. Station 200 also comprises a drain port 530, visible in FIG. 6, configured to drain liquid out of device 100. The rear-facing collection port and spacer arrangement prevents direct exposure to user urine streams and flush surges, reducing fouling risk and ensuring sensor longevity. This also avoids turbulent flow disruption during sample intake, improving test accuracy

The test assembly 230 may comprise a pump, an injector and an analyzer. The pump draws urine from collection port 218, then the injector injects the urine onto one or more test media in the cartridge, and the analyzer obtains certain property values (for example, physical/chemical properties, such as color) from the test media after they have come into contact with the urine. In an embodiment, the analyzer is an optical analyzer configured to analyze the optical properties of the test media. The injector and cartridge can move relative to each other so that the injector can open (e.g. pierce) the chamber, for example using a needle or needle-like device.

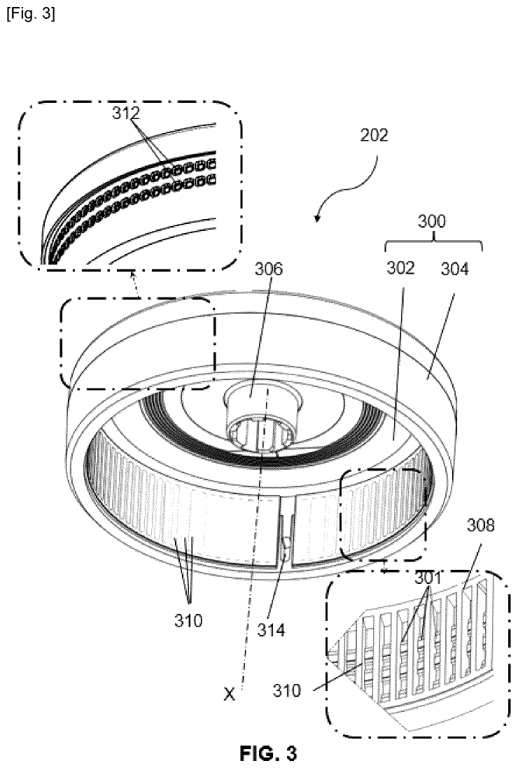

FIG. 3 shows an exploded view of cartridge 202. Cartridge 202 comprises at least one test support 301, in particular several test supports 301 configured to receive urine from the injector. Each test support 301 contains a urinary reagent which reacts in a specific way on contact with urine. The cartridge 202 comprises a rotating support 300, configured to be rotated by the station 200. During normal use of the cartridge 202 and the device 100, the test supports 301 remain attached to the rotating support and do not move relative to it.

In an embodiment, the rotating support 300 has a straight circular cylinder shape of at least 80% of a hollow cylinder shape extending annularly around an axis which is, when the cartridge 202 is mounted in the station 200, the axis of rotation X. Each test support 301 may be a test strip. The rotating support 300 may comprise an annular portion 302 and a cylindrical portion 304, which extends from a radially outer end of the annular portion 302. The cylindrical portion 304, when in use, is housed inside the annular compartment 212. Test supports 301 are positioned along cylindrical portion 304, so that they can selectively and/or successively scroll past the injector and the analyzer. For example, the test supports 301 are part of a holder 308, which comprises several chambers 310, separated from each other along a perimeter around the X axis. At least one test strip is received in a chamber 310.

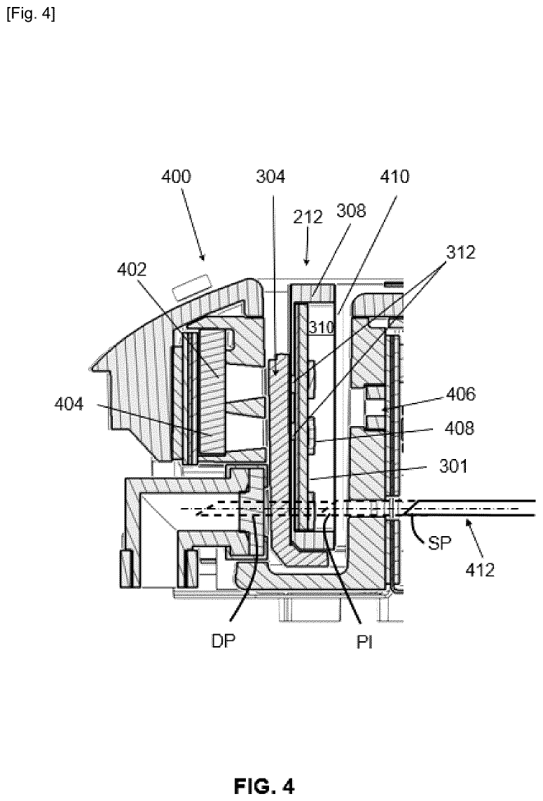

The chambers 310 are arranged side by side in the shape of a right circular cylinder of at least 80% of the circle. To allow light to pass through, the holder 308 comprises at least one opening 312 per chamber 310 (shown in the upper left zoom where the rotating support is represented as transparent). The chambers 310 are all equidistant from the axis of rotation X, so that the injector can selectively inject urine once the desired chamber is positioned at the desired location facing the injector. The injector can move towards chamber 310 and pierce a cover closing chamber 310 (visible in FIG. 4). A drain hole 314 is provided in the rotating support 300 to allow urine to drain from the injector to the outside of the device 100, via the drain port 530 located on the housing 204.

The annular portion 302 of the rotating support 300 remains outside the annular compartment 212 to reinforce the cylindrical part and/or drive the cartridge 202 in rotation. To this end, the annular portion 302 may comprise a mechanical coupling 306, which cooperates with a shaft of the station 200.

The dimensions of cartridge 202 are disclosed in documents WO′909, WO′933 and WO′80X. The maximum dimension of the device 100 transverse to the axis of rotation X is less than 15 cm, or even less than 10 cm. The maximum dimension of the device along the axis of rotation X is less than 5 cm.

FIG. 4 shows in more detail the interaction between cartridge 202 and station 200 when or after injector activation. The analyzer 400 comprises at least one light source 402, 404 (for example, two light sources) and at least one optical sensor 406. Light travels from the light source 402, 404 to the optical sensor 406, passing through the cartridge 202 and in particular the cylindrical portion 304, the opening 312 of the holder 308, the test support 301 and thus the reagent 408.

In an embodiment, the analyzer 400 is configured to measure the absorbance of a portion of the test support 301 (notably the test line and/or the control line of a strip as will be explained later). Absorbance is detected by the light source (e.g. an LED), which can pass light through the strip, and the optical sensor, which receives the spectrum at around ten wavelengths.

In an embodiment, the light sensor is a camera capable of detecting a change in color, in particular a change in color intensity, of a portion of the test support 301 (in particular the test line and/or the control line of a strip as will be explained below). The camera can detect color in RGB values, for example.

The injector comprises an injection end 412 (e.g. a needle), which can be moved between several positions, represented by dashed lines in FIG. 4. In a stand-by position SP, the injection end 412 is outside the cartridge 202 (in an innermost position), so that the cartridge 202 can rotate freely in the annular compartment 212; in an injection position IP, injection end 412 has pierced cover 410 to gain access to the inside of chamber 310 and is able to inject some urine onto test support 301; in emptying position DP, injection end 412 is able to discharge urine through drain hole 314 of rotating support 300.

In the SP position, the injector is located radially inside the annular chamber. This maximizes the radius of the annular chamber while minimizing the size of station 200.

In an embodiment of the test assembly 230 disclosed above, the skilled person will understand that each test set configured to analyze the urine sample collected through collection port 218 may be disposed within housing 204.

### Collection Port

Collection port 218 is configured to receive urine flowing by gravity over the outer surface of housing 204. Urine is collected directly on the front face 220 and rear face 222 of housing 204.

The collection port 218 is an opening configured to collect the liquid, so that the liquid can enter the urine analysis device. The collection port 218 is generally circular, with a diameter preferably between 0.3 mm and 2 mm. The diameter of the collection orifice can be chosen to maximize the volume of urine collected on the outer surface of housing 204.

As can be seen from the figures, the collection port 218 is located on the rear face 222. In this way, the collection port 218 faces the inner wall 112 of the toilet when the urine analysis device 100 is positioned in the toilet. This position allows the collection port 218 to be hidden from the user's view by the front face 220 of the housing. The front face 220 visible to the user resembles a simple, uniform pebble, as already mentioned, with no singular points or holes. It should also be noted that this position prevents the introduction of contaminants or elements that could obstruct the test assembly 230.

The collection port 218 is located on a lower part of the rear face 222. By “on a lower part of the rear face”, we mean “on the last quarter of the face along the Z direction from the lower end of the housing 204”. The lower end faces the bottom of bowl 106 when housing 204 is positioned in the bowl. The lower end is opposite the apex 550. This position corresponds to normal use. This position allows urine to be collected by gravity over most of the outer surface of housing 204.

In particular, the distance separating the collection port 218 from a lower edge of the housing 204 is less than 40 mm, preferably less than 20 mm. As illustrated, according to a particular embodiment, the collection port 218 is arranged a few millimeters above the lower edge of the housing 204. Alternatively, the collection port 218 may be located on the lower edge (the lower edge being defined when the device 100 is placed for use in a toilet).

The collection port 218 can be covered by a mesh filter. The mesh filter is, for example, oblong in shape and covers the collection port 218. The average mesh size of the filter is, for example, 20 microns. The mesh filter prevents the introduction of contaminants or elements likely to clog the test assembly 230 and filters the urine received in the collection port 218. The filter mesh can be made of metal.

### Spacer

Referring to FIGS. 5 and 6, in an embodiment, the device 100 may comprise at least one spacer 520 projecting from the rear face 222 and configured to be in contact with the bowl 106, in particular in contact with the inner wall 112 of the bowl 106. The spacer 520 is arranged on the rear face 222. In an embodiment, the spacer 520 is arranged on the rear face 222 so that the collection port 218 is at a certain distance from the bowl 106 during normal use of the device. This prevents the inner wall 112 of the bowl 106 from obstructing the inlet to the collection port 128 and/or forming a bottleneck for the flow of a mixture of urine, water and impurities from the bowl (which could otherwise be collected by the collection port).

Thanks to the at least one spacer 520, urine flows over the housing 502 (in particular on the rear face 222) towards the collection port 218 without being obstructed by the bowl 106 as could happen with a device as described in documents WO′909 and WO′944.

In an embodiment, the spacers 520 are located on the lower part of the urine analysis device 100, and more particularly on the lower part of the rear face 222, next to the collection port 218.

The 520 spacer typically comprises an edge. In use, part of the edge is in contact with the toilet bowl.

In particular, the device 100 may comprise two spacers 520, beneficially arranged on either side of the collection port 218, along the transverse direction Y. The two spacers 520 can be arranged on either side of the collection port 218, along the collection port 218. The two spacers 520 may be symmetrical, for example along a vertical plane XZ, in a normal use position of the device 100 or, for example, around the collection port 218.

The 520 spacer is described in detail in patent application EP23192265.

### FIXING

The housing 204 is designed to be placed on the inner wall 112 of the bowl 106. The housing 204 is secured by a fastening arm 500. The fastening arm 500 is configured to secure the urine analysis device 100 in the toilet bowl 106. The fastening arm 600 comprises several sections which have different functions and will be described in detail later. However, the fastening arm 600 is typically made from a single material, such as plastic, by molding.

The analysis device 100 comprises an attachment element 510 arranged on the rear face 222 of the housing. The attachment element 510 is configured to cooperate with a fastening arm 500.

The attachment element 510 is arranged at a median plane XZ of the housing 204. In other words, the attachment element 510 is arranged in a vertical plane of symmetry of the housing 204.

As can be seen from the figures, the attachment element 510 can be arranged on the upper part of the housing 204. By “on the upper part of the housing”, we mean “on the first quarter along the Z direction from the top of the housing 204”. In particular, the attachment element 510 is arranged between 15% and 25% of the rear face 222 starting from the apex 550 of the housing 204. For example, the attachment element 510 is located within a vertical band that begins approximately 15% and ends approximately 25% of the total height of the rear face 222, as measured from the upper edge 550 in the direction toward the lower end of the housing. This positional range ensures consistent placement of the fastening arm and facilitates mechanical engagement without interfering with fluid collection functions or toilet bowl geometry.

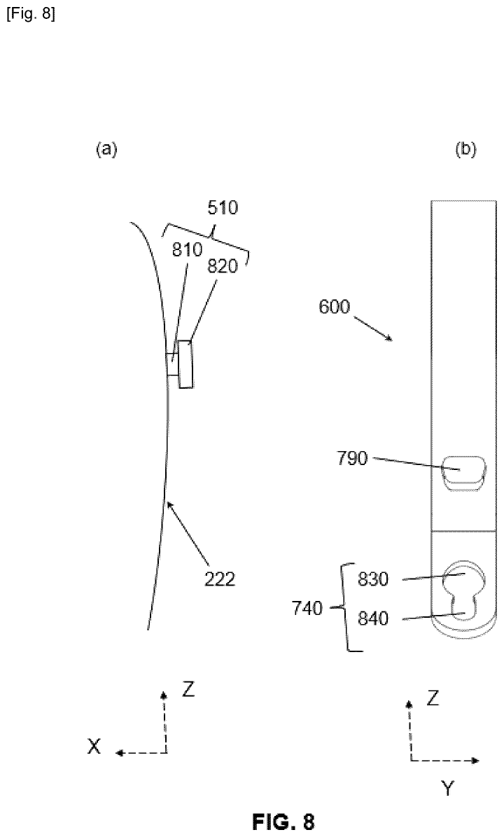

As shown in FIG. 8, the attachment element 510 may be a lug. The lug protrudes from the rear face 222 of the housing 204. In an embodiment, the lug extends from the rear face 222 along a body 810 and then a head 820 of greater transverse extent than the body 810.

In a variant not shown, the attachment element 510 is a magnet suitable for cooperating with another magnet placed on the fastening arm 500.

In a variant not shown, the attachment element 510 comprises an adhesive part suitable for cooperating with the fastening arm 500.

The proximal attachment feature, whether implemented via a lug, magnet, or adhesive, allows secure yet reversible installation. This facilitates maintenance, particularly cartridge replacement or cleaning, without requiring removal of the entire fastening assembly.

### Fastening Arms

In an embodiment, the fastening arm 500 is removable. In other words, the user can separate the fastening arm 500 from the attachment element 510 and thus from the housing. In this way, the fastening arm 500 can be easily replaced, and another form of fastening arm can be used, for example.

Fastening arm 500 can be flexible. In other words, the fastening arm 500 can be deformed by the user or by the gravity of the device 100 placed in the toilet. This makes it easier to adapt the fastening arm to different toilet shapes and ensures that the housing 204 touches the inner wall 112 of the toilet. In particular, fastening arm 500 is made of a plastic material, e.g. nylon, or a metal material, e.g. steel.

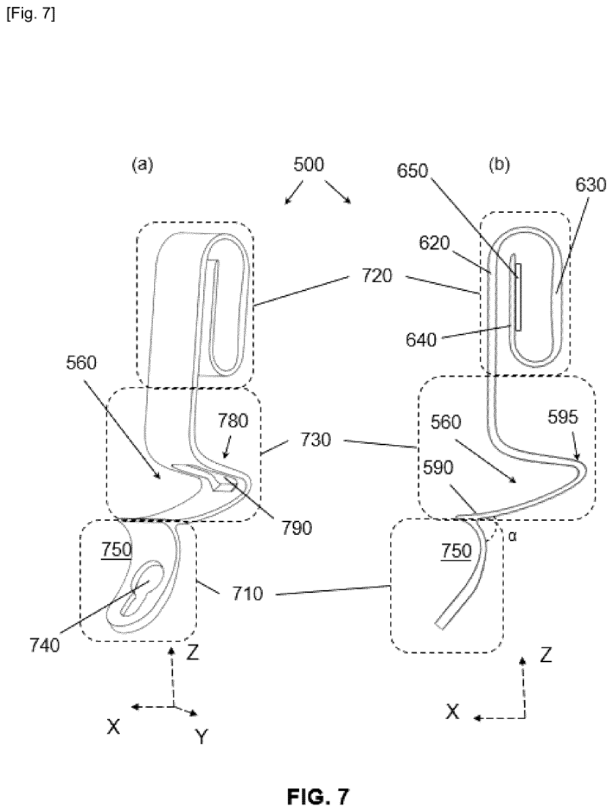

As shown in FIG. 7, the fastening arm 500 can have a flattened shape in a section transverse to the median plane XZ. This allows flexibility of the fastening arm 500 in the median plane XZ, while limiting flexibility in the transverse direction Y.

The fastening arm 500 can extend to a height along the Z axis of between 5 cm and 15 cm, in a resting configuration (i.e. neither stretched nor compressed). Fastening arm 500 may have a Y-axis width of between 1 cm and 2 cm. Fastening arm 500 has a thickness of between 1 mm and 2 mm.

### Proximal End Portion

With reference to FIG. 7, the fastening arm 500 comprises a proximal end portion 710 configured to cooperate with the attachment element 510 of the urine analysis device 100. Hereinafter, the terms “proximal” and “distal” are defined according to proximity to the urine analysis device 100.

When the attachment element 510 is a lug, the proximal end portion 710 comprises a cavity 740 designed to receive the attachment element 510. Cavity 740 may be continuous.

With reference to FIG. 8, cavity 740 has a first section 830 with a maximum transverse width greater than the width of pin head 820, and a second section 840 with a maximum transverse width less than the width of pin head 820. The second section 840 is also wider than the body 810 of the lug. So, when the user wants to assemble the fastening arm 500 with the device 100, he/she inserts the pin into the first section 830 and then slides the pin into the second section 830. As the head 820 of the pin is wider than the second section 830, the fastening arm 500 is held fast to the device 100. Conversely, when the user wishes to disengage the fastening arm 500 from the device 100, he/she slides the pin from the second section 840 towards the first section 830 to allow the head 820 of the pin to pass through the cavity 740.

### Reception Area

As shown in FIGS. 6 to 10 and 12 to 17, the proximal end portion 710 can form a receiving space 750 for the housing 204. When the fastening arm 500 cooperates with the housing 204, the receiving space 750 extends from the attachment element 510 to at least one apex 550 of the housing 204. The receiving space 750 thus extends from the rear face 222 to at least the apex 550. In an embodiment, receiving space 750 extends to the front face 220 of housing 204, as shown in FIG. 6.

The receiving space 750 is configured to at least partially receive the upper part of the rear face 222 of the housing 204, up to the apex 550. In an embodiment, the receiving space 750 is complementary in shape to the upper part of the rear face 222 of the housing 204, with, for example, a functional clearance when the device 100 is installed in the toilet bowl 106.

In an embodiment, as shown in FIGS. 6 to 8, receiving space 750 is rounded in shape, in particular complementary to the pebble shape of housing 204.

In an embodiment, the proximal end portion 710 comprises a stop 610. The stop 610 may define an end of the receiving space 750 opposite the end of the receiving space cooperating with the attachment element 510. The stop 610 is configured to block a movement of the urine analysis device 100 authorized by the fastening arm 500, in particular authorized by the connection with the attachment element 510.

### Distal End Portion

Fastening arm 500 further comprises a distal end portion 720. The distal end portion 720 is opposite the proximal end portion 710. As seen in FIG. 6, the distal end portion 720 is configured to cooperate with the toilet bowl 106.

In an embodiment illustrated in the figures, in particular FIGS. 5 to 7, the distal end portion 720 comprises a hook. In particular, the distal end portion 720 is deformable between a resting configuration, as illustrated in FIGS. 5 and 7, and a deployed configuration, as illustrated in FIG. 6. In the resting configuration, the distal end portion 720 is folded on itself; in the deployed configuration, the distal end portion 720 is configured to be hooked onto the rim 540 of the toilet bowl 106. The distal end portion 720 comprises three successive elongated parts 620, 630, 640. As can be seen in FIG. 7, in the resting configuration, the third part 640 is folded between the first part 620 and the second part 630. The three parts 620, 630, 640 are then substantially parallel to each other. As shown in FIG. 6, in the deployed position, the second part 630 and the third part 640 are spaced apart from the first part 620, with each pair of successive parts forming a substantially right angle with each other.

The third part 640 may comprise an anti-slip block 650, for example made of rubber, to improve adhesion of the fastening arm 500 to the bowl 106.

In a variant not shown, the fastening arm 500 comprises a magnet suitable for cooperating with another magnet placed in particular on the attachment element 510.

In a variant not shown, the fastening arm 500 comprises an adhesive part suitable for cooperating with the attachment element 510.

### Intermediate Portion

Fastening arm 500 further comprises an intermediate portion 730 arranged between proximal end portion 710 and distal end portion 720.

### Ramp

The fastening arm 500 comprises a water supply ramp 560 configured to direct a water flow F from a flush 670 of the toilet bowl 106 towards the front face 220 of the housing 204. In other words, the ramp 560 forms a deflector directing the flow F coming from a flush 670 of the toilet bowl 106 towards the front face 220 of the housing 204. The ramp 560 is arranged between the proximal end portion 710 and the distal end portion 720. In other words, ramp 560 forms part of intermediate portion 730.

Ramp 560 and proximal end portion 710 may form an inflection zone between them. In other words, fastening arm 500 has a change of direction between ramp 560 and proximal end portion 710. In particular, ramp 560 and proximal end portion 710 form an edge between them. In particular, the stop 610 is formed by this change of direction. In particular, as illustrated in FIG. 7 (b), the ramp 560 extends from the housing 204 towards the bowl 106, and forms an angle a of between 0° and 90° with a plane tangent to the front face 220, in particular an angle a of between 30° and 60°.

To optimize the flushing efficiency and cleaning of the urine analysis device, the ramp is configured to manipulate the flow regime of the flush water. In various embodiments, the geometry of the ramp and the orientation of its collection and transfer sections are designed to promote either laminar or controlled turbulent flow over the front face of the housing.

In embodiments where the ramp includes a smooth convex channel or diffuser grooves, the flow remains primarily laminar, which reduces splash-back and ensures even water coverage, particularly beneficial in reducing residue buildup. In alternate configurations, such as those including fins, baffles, or textured surfaces on the ramp, induced turbulence may be preferred to enhance the shear force applied to dried urine or biofilm residues.

Fluid flow simulations and testing have demonstrated that the ramp angle (such as between 30° and 60° relative to the front face) and curvature can be adjusted based on the expected flush pattern of the host toilet, allowing the system to be tuned for different bowl geometries.

The ramp 560 extends as an extension of the housing 204 and, more precisely, as an extension of or above the front face 220 of the housing 204, so that the transition between the front face 220 of the housing 204 and the ramp 560 is continuous or virtually continuous (with a functional clearance). Such an arrangement enables water to be transferred from ramp 560 to the front face 220 of housing 204. In particular, ramp 560 comprises a rear surface 570 facing the inner wall 112 of bowl 106 and a front surface 580 opposite rear surface 560. As can be seen in FIGS. 5 and 6, the front surface 580 extends in line with the front face 220 of the housing, in particular to promote the passage of flush water over the front face 220 of the housing. Alternatively (not shown), the front surface 580 can overhang the housing 204 (i.e., above the housing, thus forming a springboard towards the front face 220).

In particular, ramp 560 comprises a transfer section 590 configured to be in close proximity to housing 204. The transfer section 590 may be in contact with the housing 204 or spaced with a slight functional clearance (one or two millimeters). The transfer section 590 is configured to promote the passage of water from the flush flow F of the ramp 560 to the front face 220 of the housing 204. To this end, the transfer section 590 is flush with the front face 220 of the housing 204. In other words, the transfer section 590 and the front face 220 form a continuity of water flow between them. In other words, the plane tangent to the housing 204 at the transfer section 590 is substantially parallel to the plane tangent to the front surface 580 of the ramp 560 at the stop 610. Because of the receiving space 750, the transfer section 590 may slightly overhang the front surface 220.

The ramp 560 also comprises a collection section 595 configured to collect at least some of the water from the flush 670 to form the water flow F. The collection section 595 is configured to be close to the inner wall 112 of the toilet bowl 106. The collection section 595 is offset towards the inner wall 112 of the toilet bowl 106 in order to capture the flush water. Beneficially, the collection section 595 is in contact with the inner wall 112 of the toilet bowl 106.

The function of the collection section 595 is to collect the flush water on the ramp 560, and the function of the transfer section 590 is to transfer the water from the ramp 560 to the front face 220 of the housing 204.

In an embodiment, the intermediate portion 730 may comprise a connecting portion 780 and the ramp 560. In particular, the connecting portion 780 connects the ramp 560 to the distal end portion 720.

The connecting portion 780 comprises a passage area 790 for flush water F. The connecting portion 780 is connected to the collection section 595 of the ramp 560. Likewise, passage area 790 is connected to collection section 595. Passage area 790 allows the water flow F of the flush to pass from collection section 595 to transfer section 590 of ramp 560 (see flow F in FIG. 6). In other words, passage area 790 allows water flow F to pass from the rear of fastening arm 500 to the front of fastening arm 500.

The ramp 560 can be longitudinally convex in the median plane XZ. The ramp 560 thus forms a springboard for the water flow F of the flush.

The ramp 560 can be transversely convex, in a transverse plane YZ. The ramp 560 thus forms a channel for the flow of water from the flush 580.

The ramp 560 extends, for example, over a length of between 1 cm and 5 cm. For example, ramp 560 is wider than 1 cm.

### Diffuser

In an embodiment illustrated in FIG. 9, the ramp 560 may comprise a diffuser 910 for flush water. Diffuser 910 is configured to disperse water from ramp 560 onto the front face 220 of housing 204. Diffuser 910 comprises grooves, for example, or any shape that breaks up the flow F and disperses the flush water before the flow F flows onto the front face 220. Diffuser 910 thus makes it possible to increase the proportion of the front face 220 that is cleaned by flow F. Diffuser 910 can in particular be positioned at the level of transfer section 590.

### Fin

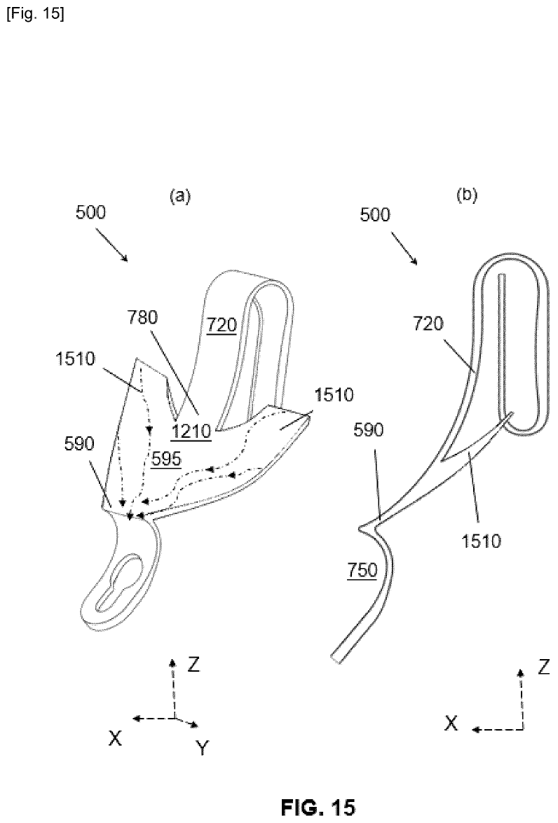

In an embodiment, as illustrated in FIG. 15, the ramp 560 may have at least one lateral fin, in particular two lateral fins 1510. Each lateral fin 1510 extends in particular transversely from intermediate portion 730. Each side fin 1510 is configured to capture flush water flowing next to the fastening arm 500 and redirect it towards the transfer section 590 and thus towards the housing 204.

### Ramp and Connecting Portion in Series

FIGS. 5 to 11 show an example of a urine analysis system as described in this description, referred to as a “series” system.

As can be seen from the figures, ramp 560 is arranged here between proximal end portion 710 and connecting portion 780.distal end portion 720, connecting portion 780, ramp 560 and proximal end portion 710 are arranged successively, in other words “in series”.

In an embodiment shown in FIG. 7, ramp 560 and connecting portion 780 form a V-shape. In other words, ramp 560 and connecting portion 780 form an inflection zone between them. In other words, fastening arm 500 has a change of direction between ramp 560 and connecting portion 780.

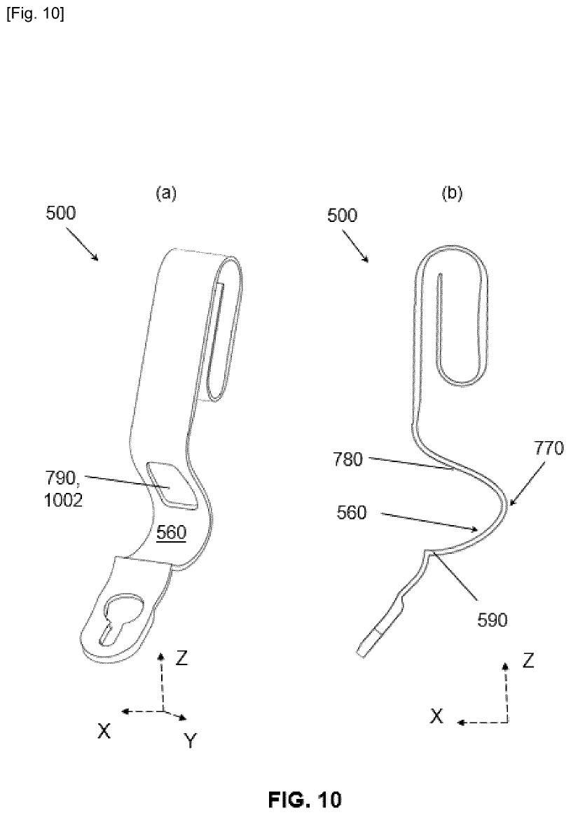

In a variant illustrated in FIG. 10, the ramp 560 and the connecting portion 780 form a U-shape. In other words, the collection section 595 has a rounded shape.

Two variants of the connecting portion 780 are described below.

### Opening Variant

In a variant of the series arrangement shown in FIGS. 6 to 10, the passage area 790 is defined by an opening 1002 in the connecting portion 780. The opening 1002 allows water to pass from the flush water flow F to the ramp 560. Opening 1002 extends at least as far as collection section 595.

The opening 1002 has a transverse width greater than 50%, in particular greater than 75%, of the transverse width of the connecting portion 780. The opening has a length greater than 50%, in particular greater than 75%, of the transverse width of the connecting portion 780.

Here, opening 1002 has a substantially rectangular shape, with rounded edges. However, other shapes can be envisaged to allow water to pass through

### Narrowing Variant

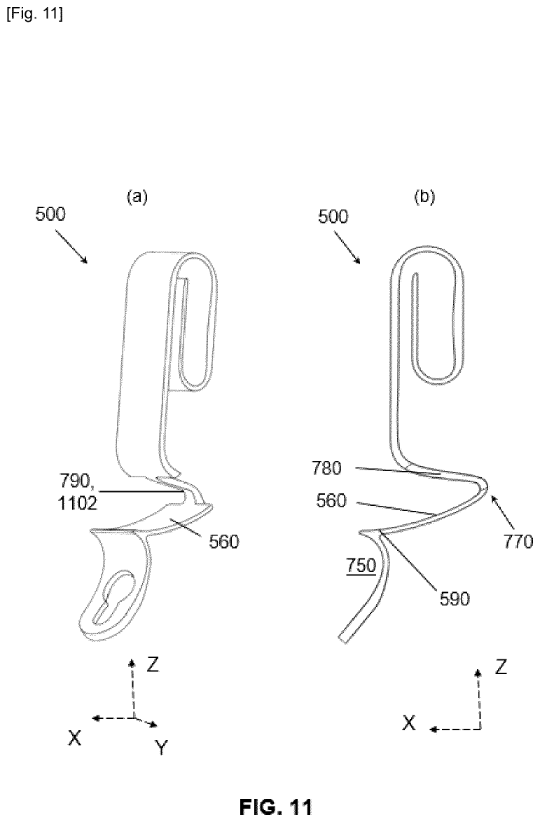

In a variant of the series embodiment shown in FIG. 11, the passage area 790 is defined by a narrowing 1102 in cross-section at the connecting portion 780. The narrowing 1102 allows the passage of water from the water flow F on either side of the narrowing towards the ramp 560. In particular, the narrowing 1102 is a reduction in the width of the connecting portion 780 relative to the ramp 560. The narrowing extends at least as far as the collection section 595.

The narrowing 1102 has a transverse width of less than 50%, in particular less than 75%, of the transverse width of the connecting portion 780. The constriction has a length greater than 50%, in particular greater than 75%, of the transverse width of the connecting portion 780.

The narrowing 1102 has a substantially rectilinear shape, centered in the middle of the connecting portion 780. However, other shapes can be envisaged to allow the passage of water.

### Ramp and “Parallel” Connecting Portion Embodiment

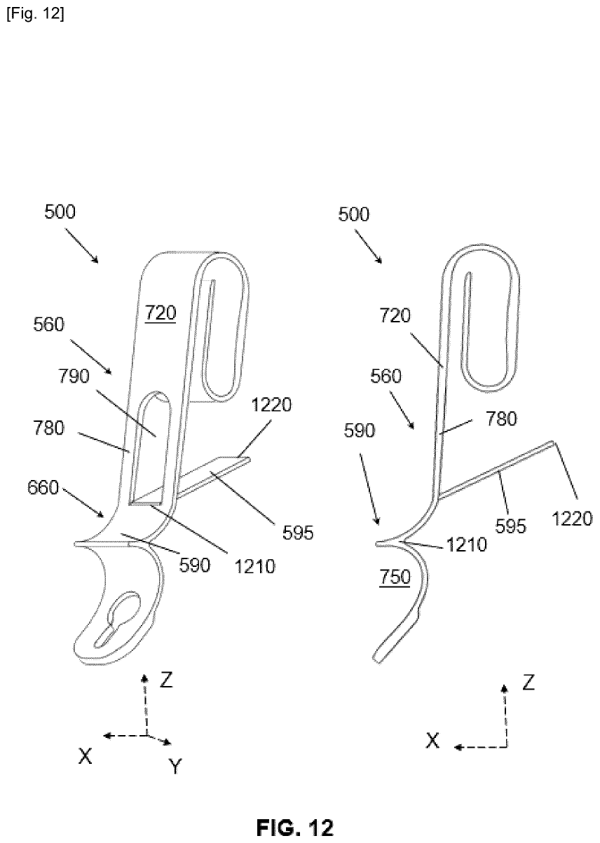

FIGS. 12 to 16 show a “parallel” version of the urine analysis system described herein.

As can be seen from the figures, the connecting portion 780 is arranged here between the transfer section 590 of the ramp 560 and the distal end portion 720. The collection section 595 of the ramp 560 extends from a junction 1210 between the connecting portion 780 and the transfer section 590. In particular, collection section 595 extends towards the inner wall 112 of bowl 106.

The collection section 595 comprises a free end 1220 on the opposite side to the junction 1210. The free end 1120 faces the inner wall 112 of the bowl 106, which is in contact with the inner wall 112, for example. In other words, collection section 595 forms a fin extending towards bowl 106.

In this embodiment, connecting portion 780 and collecting section 595 each extend from junction 1210, i.e. connecting portion 780 and collecting section 595 are “in parallel”.

### Parallel Aligned Variant

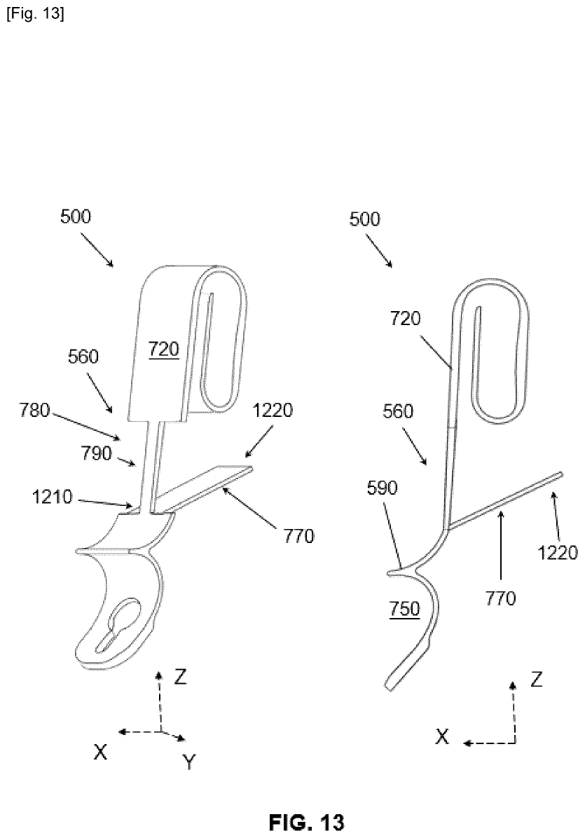

In a variant of the parallel embodiment illustrated in FIGS. 12 and 13, to enable water to pass from the collection section 595 to the transfer section 590, the connecting portion 780 comprises a passage area 790 (from which the collection section 595 of the ramp 560 extends in particular, in the opposite direction to the transfer section 590). The collection section 595 of the ramp 560 protrudes from the passage area 790 towards the trough 106. Opening sub-variant

In a sub-variant of the previous variant, illustrated in FIG. 12, the passage area 790 is defined by an opening. The opening allows water to pass from the collection section 595 through the opening to the transfer section 590. The opening extends at least as far as junction 1210

The opening has a transverse width greater than 50%, in particular greater than 75%, of the transverse width of the connecting portion 780. The opening has a length greater than 50%, in particular greater than 75%, of the transverse width of the connecting portion 780.

Here, the opening is shaped like an arch. However, other shapes can be envisaged to allow water to pass through.

### Shrinkage Sub-Variant

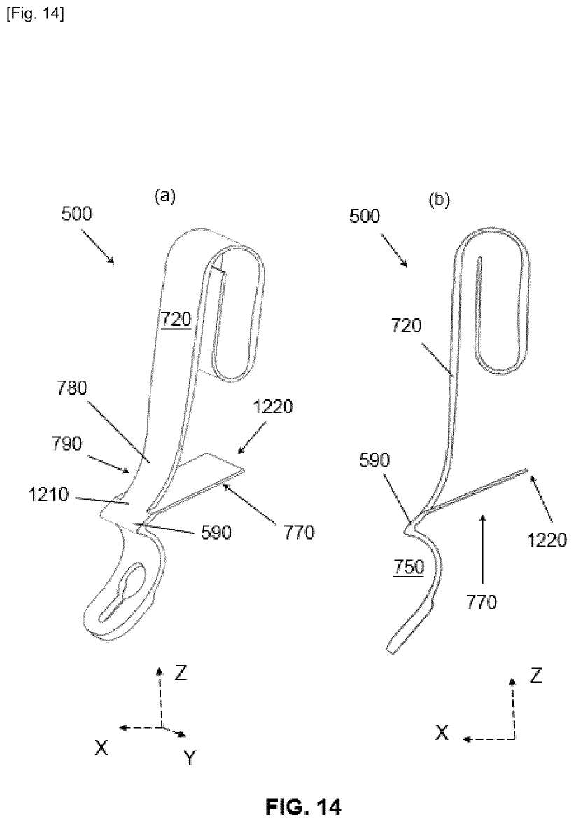

In an embodiment illustrated in FIGS. 13 and 14, the passage area 790 is defined by a cross-sectional constriction at the connecting portion 780. The constriction allows the passage of flush water on either side of the constriction from the collection section 595 to the transfer section 590. In particular, the narrowing is a reduction in the width of the connecting portion 780 relative to the ramp 560. The narrowing extends at least as far as junction 1210.

The narrowing has a transverse width of less than 50%, in particular less than 75%, of the transverse width of the connecting portion 780. The narrowing has a length greater than 50%, in particular greater than 75%, of the transverse width of the connecting portion 780.

Referring to FIG. 13, the constriction can have a substantially rectilinear shape, centered in the middle of the connecting portion 780

In a variant shown in FIG. 14, the narrowing is a progressive reduction in cross-section from the distal end portion 720 to the junction 1210, so that the cross-section of the connecting portion 780 is minimal at the junction 1210 to allow water to pass through on either side.

Obviously, other shapes can be envisaged to allow water to pass through.

### Ramp Design

FIG. 15 shows an example of the “winged ramp” urine analysis system described herein.

As can be seen in this figure, the ramp 560 is arranged directly between the proximal end portion 710 and the distal end portion 720. Thus, in this embodiment, the intermediate portion 730 does not comprise a connecting portion as defined above.

The collection section 595 of the ramp 560 comprises one or more fins 1510 extending laterally from a central portion of the ramp 560. In particular, the collection section 595 comprises two fins 1510 extending on either side of the central portion of the ramp 560. In addition, fins 1510 can extend towards the wall of the bowl, so as to come into contact therewith.

Each fin 1510 is configured to capture flush water flowing past the fastening arm 595 and redirect it to the transfer section 590 and thus to the housing 204.

### Funnel Version

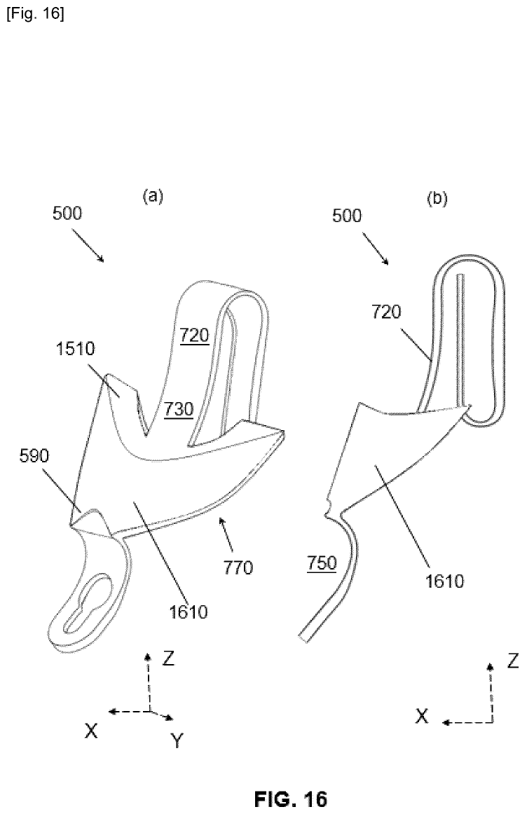

In a variant of the winged ramp design shown in FIG. 16, the collection section 595 comprises a funnel 1610 that extends around the central part of the ramp 560. The funnel can be formed in part by fins 1510. The funnel channels the flow of water F and directs it entirely towards the transfer section 590.

### Single-Boom Design

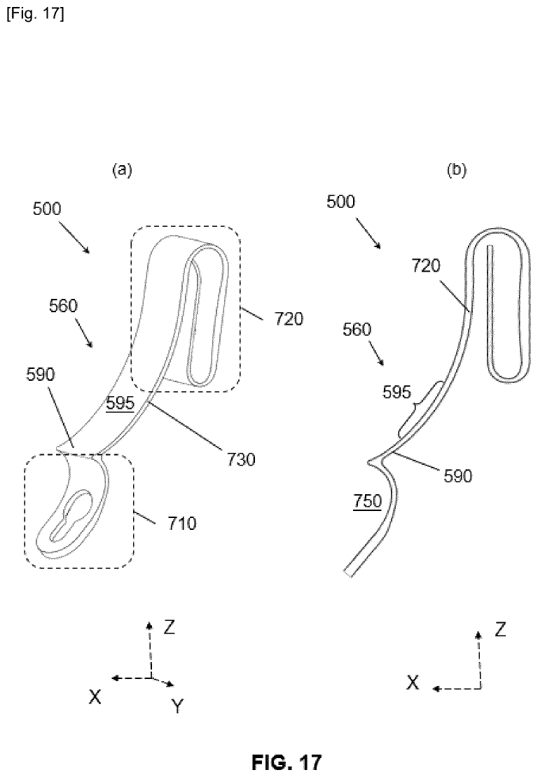

FIG. 17 shows a single-ramp version of the urine analysis system described herein.

As can be seen in this figure, the ramp 560 is arranged directly between the proximal end portion 710 and the distal end portion 720. Thus, in this embodiment, the intermediate portion 730 does not comprise a connecting portion as defined above.

The collection section 595 of ramp 560 is formed by a part of ramp 560 located close to, or even in contact with, the inner wall 112 of bowl 106. To this end, ramp 560 may be rounded in shape. In particular, ramp 560 may have a convex shape to bring the collection section 595 closer to the inner wall 112 of bowl 106.

In this way, a flush water stream flowing transversely over the inner wall 112 is captured by the ramp 560. According to another bowl architecture, the collection section 595 of the ramp 560 is configured to capture water from two flush water flows meeting at the fastening arm 500.

The transfer section 590 is configured to be flush with the front face 220 of the housing 204 and allow water to be transferred from the ramp 560 to the front face 220.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A urine analysis assembly comprising:
a urine analysis device configured to be placed entirely within a toilet bowl and comprising a housing, the housing comprising a front face for receiving a stream of urine directly from a user urinating into the toilet bowl, and a fastening arm configured to position the urine analysis device entirely within the toilet bowl, the fastening arm comprising a water supply ramp configured to direct a flow of water from a flush of the toilet bowl towards the front face of the housing.

2. The urine analysis assembly according to claim 1, wherein the ramp extends as an extension of the front face of the housing of the urine analysis device or overhangs the front face.

3. The urine analysis assembly according to claim 1, wherein the ramp has at least one fin configured to extend towards the toilet bowl.

4. The urine analysis assembly according to claim 1, wherein the ramp comprises a diffuser for flush water, the diffuser being configured to disperse water from the water flow of the ramp onto the front face of the housing.

5. The urine analysis assembly according to claim 1, wherein the fastening arm comprises a proximal end portion configured to cooperate with the housing.

6. The urine analysis assembly according to claim 1, wherein the ramp comprises a transfer section close to the housing and configured to promote the passage of water from the water flow of the ramp towards the front face of the housing.

7. The urine analysis assembly according to claim 6, wherein the transfer section is flush with the front face of the housing.

8. The urine analysis assembly according to claim 1, wherein the ramp comprises a collection section configured to collect at least a portion of the flush water.

9. The urine analysis assembly according to claim 1, wherein the fastening arm further comprises a distal end portion configured to cooperate with the toilet bowl and an intermediate portion arranged between the proximal end portion and the distal end portion, the intermediate portion comprising a connecting portion and the ramp.

10. The urine analysis assembly according to claim 9, wherein the connecting portion comprises a passage area allowing the passage of water from the water flow.

11. The urine analysis assembly according to claim 10, wherein the passage area is defined by an opening in the connecting portion.

12. The urine analysis assembly according to claim 10, wherein the passage area is defined by a cross-sectional narrowing at the connecting portion.

13. The urine analysis assembly according to claim 9, wherein the ramp is arranged between the proximal end portion and the connecting portion.

14. The urine analysis assembly according to claim 9, wherein the ramp comprises a transfer section close to the housing and configured to promote the passage of water from the water flow of the ramp towards the front face of the housing, and wherein the connecting portion is arranged between the transfer section and the distal end portion.

15. The urine analysis assembly according to claim 14, wherein the ramp comprises a collection section configured to collect at least a portion of the flush water, wherein the collection section of the ramp extends from a junction between the connecting portion and the transfer section, the collection section comprising a free end on the opposite side to the junction.
